(19) United States
(12) Patent Application Publication
(10) Pub. No.: US 2019/0247675 A1
(43) Pub. Date: Aug. 15, 2019

(54) PROTECTIVE LIGHTING METHOD

(71) Applicant: MYOLITE, INC., Jupiter, FL (US)

(72) Inventors: Jerome A. Legerton, Jupiter, FL (US); Steven W. Carlin, Lake Forest, CA (US)

(21) Appl. No.: 16/392,506

(22) Filed: Apr. 23, 2019

Related U.S. Application Data
(63) Continuation of application No. 14/033,335, filed on Sep. 20, 2013.
(60) Provisional application No. 61/703,424, filed on Sep. 20, 2012.

(51) Int. Cl. A61N 5/06; F21S 4/20; F21K 9/23; F21S 8/04; F21V 19/02; F21K 9/65; H05B 33/08

(52) U.S. Cl. CPC A61N 5/0613; F21S 4/20; F21K 9/23; F21S 8/04; F21Y 2115/10; F21K 9/65; H05B 33/086; A61N 2005/0667; F21V 19/02

(57) ABSTRACT

The present application is directed to a method for the regulation of the development of ocular refractive errors, comprising: controlling at least one light source using a processor, the at least one light source emitting an electromagnetic radiation variable with respect to one or more of a direction, an illuminance, a retinal area, an amplitude, a wavelength, and a spectral output; and regulating the at least one light source and producing a spectral power distribution at a plane of an eye.

### REFERENCE TO RELATED APPLICATIONS

This application is a continuation of U.S. patent application Ser. No. 14/033,335, filed Sep. 20, 2013, which claims priority to U.S. Provisional Application No. 61/703,424, filed on Sep. 20, 2012, the contents of which are incorporated herein by reference in their entireties.

### FIELD OF THE INVENTION

The present invention relates generally to refractive therapy, and more particularly, to lighting systems for the regulation of the development of refractive error.

### BACKGROUND OF THE INVENTION

Refractive correction can be achieved through use of spectacle lenses, contact lenses, corneal refractive surgery and intraocular lens implantation. Contact lenses have evolved from non-gas-permeable rigid lenses which contact the sclera and vault the cornea to corneal contact lenses made of gas permeable products, and then to corneal-scleral contact lenses made of hydrogel materials. Hybrid lenses were created to provide the improved optics of rigid lenses with the comfort of soft lenses. Hybrid lenses are typically configured to have a central rigid zone joined at a radial junction to a peripheral hydrogel zone. Composite lenses have a full soft layer and those having only an annulus of soft posterior to the rigid layer have been anticipated.

Hybrid lenses of this configuration enjoy commercial success with limitations due to the separation of the two materials at their radial junction, lens flexure and tear stagnation due to a circumferential sealing of the lens against the underlying eye. Advanced manufacturing processes and ultra high gas permeable materials have stimulated a resurgence of fully rigid scleral lens designs.

Rigid, soft and composite lenses have been used or envisioned for corneal reshaping or corneal refractive therapy. Corneal refractive therapy by means of the peripheral defocus appears to have value in changing the optics of the cornea with a concomitant benefit in regulating the development of the refractive error of the eye. Recent research points to the role of light or illumination in the regulation of the development of refractive errors of the eye.

Smith and co-workers reported results of exposure of the eyes of primates to peripheral illumination as an opposite to form deprivation and found that eyes having peripheral retinal illumination exposure experienced less axial length growth than those having a lower level of illumination. (E. L. Smith III, L. Hung and J. Huang, Protective Effects of high ambient lighting on the development of form-deprivation myopia in rhesus monkeys, IOVS, December 2011, http://www.iovs.org/content/53/1/421.abstract). Further, they found these effects to be regional indicating the possible specificity of peripheral illumination.

The work of Wildsoet in 2002 provided early evidence to the importance of light (including the wavelength of the light) for limiting the growth of eye length. (See C. Wildsoet, Recent insights from animal myopia research, Beijing Seminar, November 2002).

The work of Rucker and Wallman in 2008 demonstrates the role of the wavelength of light on choroidal thickness and eye elongation in dim illumination. (See F. J. Rucker, J. Wallman, Cone signals for spectacle-lens compensation: Differential responses to short and long wavelengths, 2008).

The work of J. Guggenheim and co-workers demonstrated that time spent outdoors was predictive of incident myopia independently of physical activity level. The greater association observed for time outdoors suggests that the previously reported link between sports/outdoor activity and incident myopia is due mainly to its capture of information relating to time outdoors rather than physical activity. This suggests the role of outdoor illumination is protective to the development of myopia. (J. Guggenheim Time Outdoors and Incident Myopia in Childhood. IOVS, May 2012, Vol. 53, No. 6).

The work of J. Siegwart and co-workers demonstrated that a group of tree shrews exposed to elevated fluorescent light levels for eight hours per day developed 47 percent less myopia than a control group exposed to normal indoor lighting, even though the images were neither more nor less blurry. (J. Siegwart, Moderately elevated light levels slow form deprivation and minus lens induced myopia development in tree shrews. Paper presented IOVS, May 8, 2012).

The work of J. Sherwin and co-workers measured a statistically significant inverse relationship in humans between conjunctival ultraviolet autofluorescence (UVAF), a biomarker of outdoor light exposure, and the prevalence of myopia. They suggest that the marker is a stronger indicator of the protective factor than time outdoors alone. The marker is the result of ultraviolet light exposure. Their work suggests that the level of ultraviolet light is important. (J. Sherwin, The association between time spent outdoors and myopia using a novel biomarker of outdoor light exposure. IOVS. 2012; 53(8):4363-4370.).

Researchers have identified the presence of a lower blood serum level of Vitamin D in individuals who develop myopia. (D. O. Mutti, Vitamin D receptor (VDR) and group-specific component (Vitamin D binding protein) polymorphisms in myopia, The Association for Research in vision and Ophthalmology, February 2011). Exposure to ultraviolet wavelengths in the electromagnetic spectrum is known to stimulate Vitamin D in the body.

According to Holick, approximately 22 minutes of sunlight near midday will produce 1.5 minimal erythema doses (MED) of UVB radiation exposure which is enough to induce a pronounced temporary increase in vitamin D concentration (Holick, 1985). Current “Full Spectrum” fluorescent lamps that produce UV radiation would require 30 hours to produce an equivalent level when operated at ceiling height.

Contemporary health science holds ultraviolet exposure to be detrimental with specific concern for skin cancer, retinal degeneration and cataracts. Health care professionals generally recommend protection from UV exposure by avoiding extended periods in sun light, use of eyewear with ultraviolet absorbers and the use of sun screening products to protect skin. Cultural preferences exist in ethnic groups which include having light skin with the concomitant pattern of protecting the eyes and body from sun light and avoiding time outdoors.

The increase in incidence and resultant prevalence of myopia in the developed world and most particularly in Asia presents a problem of epidemic proportion. The changes in life-style, living conditions and activity preferences often prevent the ability to engage in outdoor activities. Educational, vocational and avocational demands and habits generate a set of circumstances which replace the available time for exposure to ambient outdoor light. Further, the needs to conserve energy indoors may have an ongoing effect in reducing the ambient light levels inside homes and buildings.

Research supports that the mechanism for the development of refractive error is multivariate. As such, preventive therapeutic strategies are anticipated which incorporate multiple therapeutic components.

At least two ocular components are known to change as part of refractive error development. The first is the crystalline lens geometry and the second is the vitreous chamber depth of the eye. In the normal process these anatomic components change in concert with each other to render the optical system of the eye appropriate for the vitreous chamber depth of the eye. It is also known by those skilled in the art that the equatorial diameter of the eye may vary relative to the axial length of the eye. Eyes which manifest myopia are often found to be more prolate in geometry and having an equatorial diameter which is smaller relative to their axial length than eyes manifesting hyperopia.

The local or regional changes in the anatomy of the eye resulting from exposure to various wavelengths of light involve at least two measurable components. The first is a change in choroidal thickness and the second is eye elongation. Myopia is associated with a thinning of the choroid and elongation of the vitreous chamber of the eye.

The role of peripheral defocus and peripheral illumination are believed to have an influence on the local growth factors which influence the shape of the crystalline lens, the equatorial diameter and the axial length of the eye.

Neitz et al. have developed a method and apparatus for limiting the growth of eye length. (See U.S. Patent Publication Nos. (See U.S. Patent Publication No. 2011/0313058). Although Neitz teaches the importance of wavelength modulation, the intervention is limited to filters of red light. (See, e.g., claim 17). Such filters fail to modulate brightness above an ambient level. They also fail to add the component of near visible ultraviolet light.

Full spectrum lamps have been marketed which claim to replicate outdoor lighting along with a number of claimed benefits. While the Correlated Color Temperature may fall within a level found in the range of daylight, the spectral power distribution most often has spikes and fails to represent outdoor daylight. The use of a Full Spectrum Index (FSI) has been suggested as a preferred means to calculate the equal energy across the full spectrum by use of the measured Spectral Power Distribution (SPD)

The FSI fails to reflect the importance of modulating the SPD for the purpose of refractive error regulation. Research indicates that ultraviolet light may play an important role in regulating myopia and further, the longer wavelength red light may be detrimental, most particularly when the lighting condition is dim. A preferred protective lighting system is best described by the spectral power distribution and illuminance at the plane of the eye. Such a system is impacted by architectural features and filters on light sources, the distance from the source to the eye and the reflective nature of the surface in proximity to the eye.

### SUMMARY OF THE INVENTION

In view of the above, there exists a need for a protective lighting system comprising one or more light sources and architectural features which produce a pre-determined spectral power distribution and illuminance at the plane of the eye.

Embodiments of the present invention provide devices and methods for lighting systems intended for the regulation of refractive error. Such regulation can be achieved by incorporation of light sources and architectural elements which can be configured in a directional manner and can vary in the spectral power distribution and illuminance of the radiation. Various embodiments provide illumination at the plane of an eye to produce the optimum spectral quality and quantity of light. Depending on the embodiment, this may be achieved with or without the concomitant provision of vision correction or corneal refractive therapy and with or without the use of contact lenses.

Various embodiments of the present invention set forth light fixtures and elements having illumination modulating components for the purpose of regulating the change in the ocular components which result in the presence or absence of refractive error. While the prior art (Neitz) teaches filtering red light, embodiments of the invention teach radiating with the blue end and near-visible short wavelength ultraviolet light, along with an adequate amplitude of light with consideration for the energy efficiency (Efficacy) of the system.

One embodiment of the present invention comprises customizable, modular LED lights as set forth in U.S. patent application Ser. No. 12/709,384 to Carlin, the content of which is incorporated herein by reference in its entirety. Carlin teaches, inter alia, an LED tube light with an external driver which may allow drivers with a range of power output and LED strips which may be configured with a variety of individual diodes. The selection of the diodes and phosphors provides the predetermined spectral power distribution and illuminance with the optimized lumens per watt (Efficacy).

According to an embodiment of the present invention, a protective lighting system comprises: an electromagnetic radiation source comprising an LED light source that directs one of its on axis or off axis electromagnetic radiation through the crystalline lens of the eye and to a desired retina area of an occupants eye; wherein the electromagnetic radiation source includes spectral characteristics present in outdoor light.

### BRIEF DESCRIPTION OF THE DRAWINGS

FIG. 1 is a diagram illustrating a lighting system with at least one electromagnetic radiation that directs one of its “on” axis or “off” axis electromagnetic radiation to a desired retina area of a person, in accordance with an embodiment of the invention.

FIGS. 2A-2C are diagrams illustrating a lighting system having at least one electromagnetic radiation source that is directed through a crystalline lens to a pre-determined retinal area of a person, in accordance with an embodiment of the invention.

FIG. 3 is a diagram illustrating a spectral power distribution (SPD) of an embodiment of the present invention.

FIGS. 4A and 4B are diagrams illustrating a light tube housing a plurality of LEDs in accordance with an embodiment of the present invention.

FIG. 5 is a flowchart illustrating a method of designing an optimum lighting system, in accordance with an embodiment of the invention.

FIG. 6 is a flow diagram illustrating an example of a computing module for implementing various embodiments of the disclosure.

### DETAILED DESCRIPTION

In the following paragraphs, the present invention will be described in detail by way of example with reference to the attached drawings. Throughout this description, the preferred embodiment and examples shown should be considered as exemplars, rather than as limitations on the present invention. As used herein, the “present invention” refers to any one of the embodiments of the invention described herein, and any equivalents. Furthermore, reference to various feature(s) of the “present invention” throughout this document does not mean that all claimed embodiments or methods must include the referenced feature(s).

Embodiments of the invention provide an electromagnetic radiation system disposed on or within a space intended for occupants (e.g., humans) and including at least one electromagnetic radiation source that is directed toward the retina or passes through the eye off of the visual axis. By way of non-limiting example, the electromagnetic radiation source may comprise light emitting diodes (LEDs), incandescent lighting, fluorescent lighting, compact fluorescent lighting, metal halide lighting, ceramic metal halide lighting, mercury vapor lighting, xenon lighting, or other sources used in producing artificial light or transmitting outdoor light into the occupied space. The electromagnetic radiation system is configured to produce radiation having a predetermined: (i) amplitude, (ii) spectral power distribution, and (iii) minimal erythema doses from the ultraviolet spectral contribution, at the plane of the eye.

The Color Rendering Index (CRI) is used to design and communicate lighting systems which have a spectral quality that renders the color of objects to be optimum. An equal energy spectrum demonstrates a higher CRI than a source which has spikes and valleys in its spectral power distribution. An embodiment of the invention provides ultraviolet radiation and reduces the long wavelength portion of the visible spectrum. The ultraviolet radiation is expected to be compromised in its color rendering index while providing a protective factor for the development of myopia.

Energy efficiency is a critical component of a modern lighting system and is rated as; Efficacy=lumens/watt. Some embodiments of the protective lighting system includes a specification for Efficacy in an effort to increase the utilization of the system for its preventive value without generating an economic barrier to its adoption.

Current LED (Light Emitting Diode) sources are anticipated to provide the greatest efficacy for the protective lighting system. They may comprise single spectral output LED or an LED mix including LEDs with different spectral output for the purpose of tuning the spectral power distribution. The LED mix may be tunable and may be modulated by a variable power source, or variable attenuation or vignetting in the outer tube, or architectural structures external to the LED element. In certain embodiments, the lighting system may be controlled by a computer program product which may in turn be coupled to sensors remote to the occupants in the environment or in the plane of the eye of the occupants.

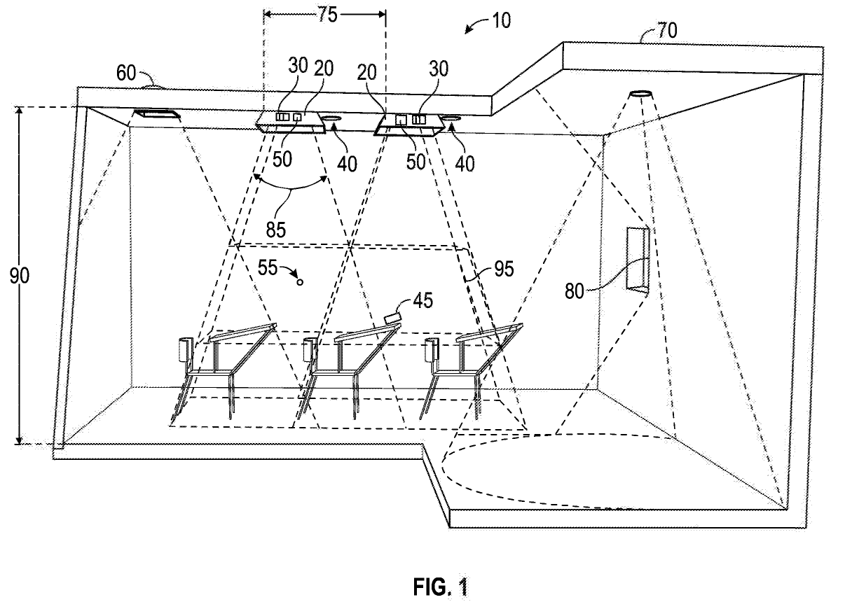

Referring to FIG. 1, a lighting system 10 having at least one electromagnetic radiation source 20 will now be described. Specifically, the electromagnetic radiation source might comprise a light source 20 that directs one of its on axis or off axis electromagnetic radiation to the plane of a desired retina area of a person seated in a classroom. The power source for powering the light can comprise any suitable power source including a conventional power outlet, batteries 30, an electrical generator, etc. The system 10 further comprises an antenna 40 for receiving signals (e.g., from remote control 45) and a processor 50 in order to control, e.g., an illuminance, correlated color temperature and spectral band width of the light produced by the light source. The electromagnetic radiation source 20 can comprise LEDs, incandescent lighting, fluorescent lighting, compact fluorescent lighting, metal halide lighting, ceramic metal halide lighting, mercury vapor lighting, xenon lighting, or other sources used in producing artificial light or transmitting outdoor light into the occupied space. For example, outdoor light may be controllably transmitted through a skylight 60, solar tube 70, or window 80, by using a manually or remotely controllable shutter. The lighting system 10 is configured to produce radiation having a predetermined: (i) amplitude, (ii) spectral power distribution, and (iii) minimal erythema doses from the ultraviolet spectral contribution, at the plane of the eye.

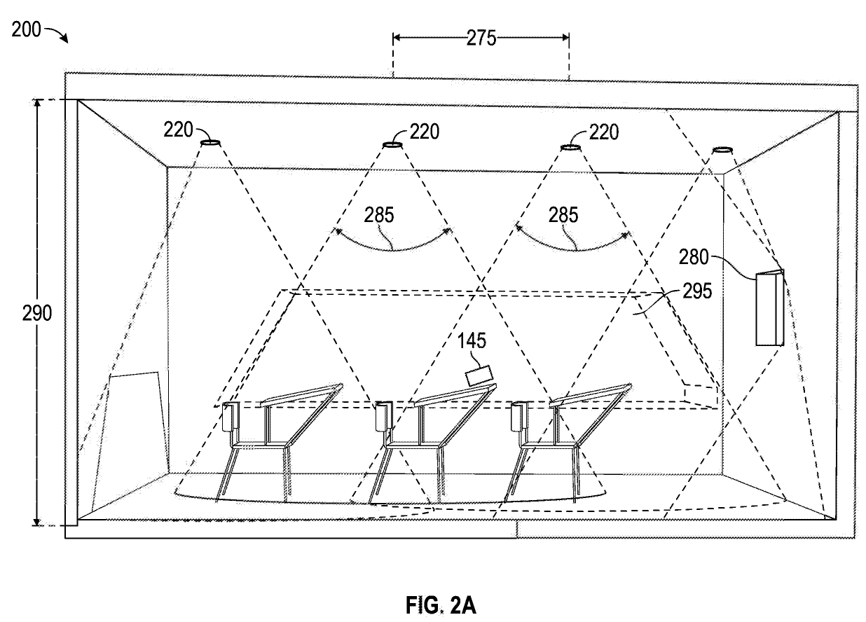

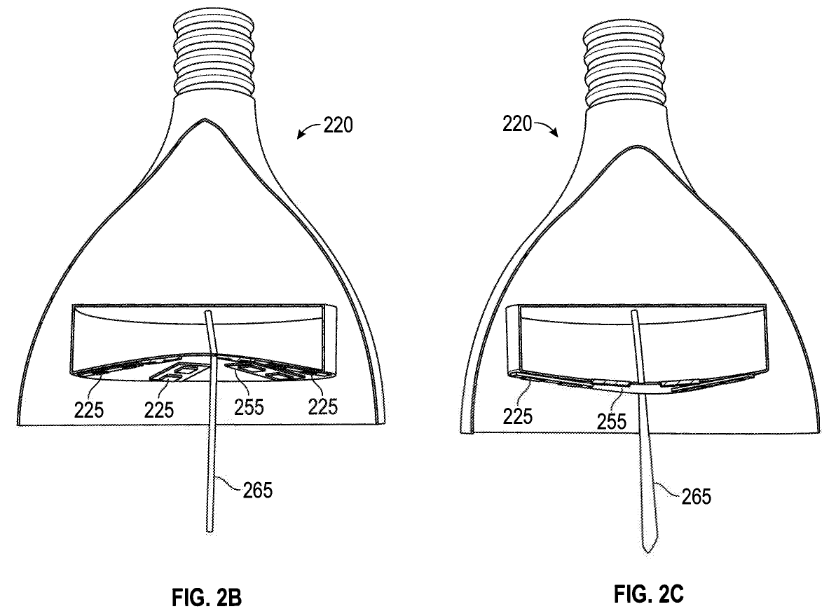

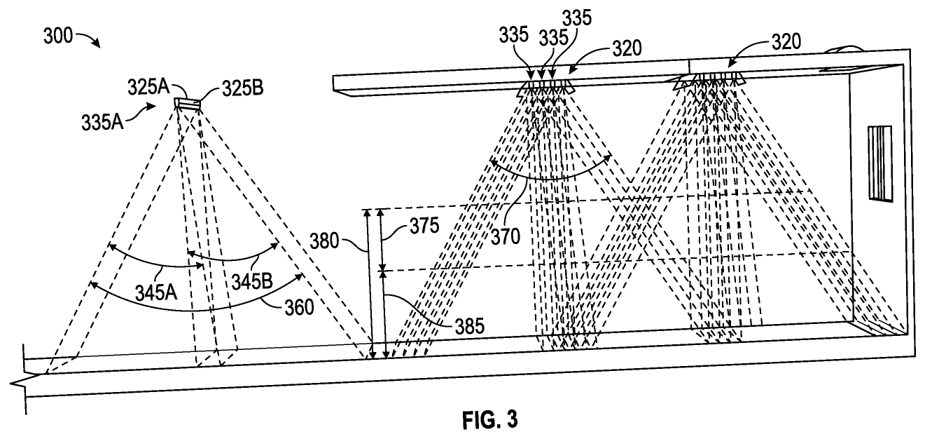

With further reference to FIG. 1, the electromagnetic radiation source 20 can be designed to have spectral characteristics present in outdoor light. As stated, the electromagnetic radiation source 20 is programmable with respect to direction, illumination, retinal area, amplitude, wavelength, and/or spectral property. Alternatively, the electromagnetic radiation source 20 may include a predetermined direction, illumination, retinal area, amplitude, and/or wavelength/spectral character. The electromagnetic radiation source 20 may include a number of light tubes 35, as shown in FIGS. 1 and 3, or may comprise a ring of LEDs 220, as shown in FIG. 2. The electromagnetic radiation source and light elements (light tubes, LEDs) can be any suitable geometric form. In addition, the source 20 may be varied in its position or size, and any number of sources 20 may be employed. In the illustrated embodiment, there are two electromagnetic radiation sources 20 separated by a predetermined distance 75, transmitting electromagnetic radiation within a predetermined angle 85, thereby creating an optimal zone 95 of electromagnetic radiation. The distance between the floor and ceiling is indicated as element 90.

With continued reference to FIG. 1, designing an optimum lighting system initially entails measuring the ambient illumination to determine the contribution from architectural structures and incident outdoor lighting through windows 80, skylights 69, tubes 70 or other means of transmitting outdoor light. The next steps might entail (i) calculating the needed spectral power distribution to be delivered via supplemental light sources 20, and (ii) determining the location for placement of the required supplemental light sources 20. The supplemental lighting sources are then selected to provide the required SPD, illuminance and MED for the pre-determined eye-planes in the room. Optionally, the light sources 20 may be programmably controlled by way of one or more algorithms residing in processor 50. In operation, the system is installed with the respective sensors (e.g., light sensor 55), sources (e.g., light sources 20) and programmable controllers (e.g., remote control 45).

Referring to FIGS. 2A-2C, another lighting system 200 having at least one electromagnetic radiation source 220 will now be described. Lighting system comprises at least one electromagnetic radiation source 220 that is directed through a crystalline lens to a pre-determined retinal area of a person. Similar to the above-described system 10, system 200 can further comprises batteries for powering the light, an antenna for receiving signals (e.g., from remote control 145) and a processor for controlling the light. In the illustrated embodiment, the electromagnetic radiation source comprises an LED light 220 comprising a ring of LEDs that directs one of its on axis or off axis electromagnetic radiation to the plane of a desired retina area of a person seated in a classroom. As depicted in FIG. 2B, the LED light 220 can include a ring of LEDs 225. Like the system 10 of FIG. 1, system 200 may include batteries for powering the LEDs, an antenna for receiving signals (e.g., from a remote control) and a processor including one or more algorithms for controlling the LEDs. Additionally, outdoor light may be controllably transmitted through a source of outdoor light such as window 280 using a remotely controllable shutter.

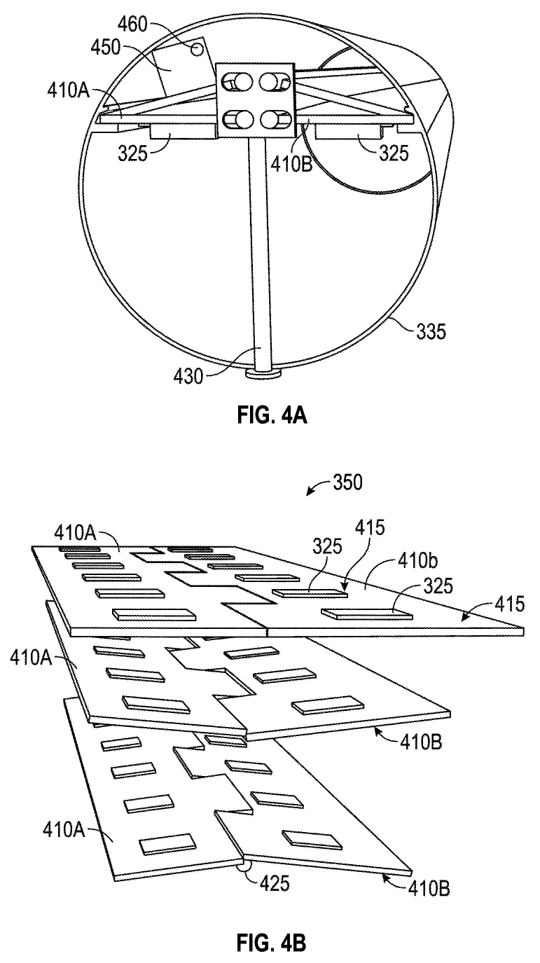

With reference to FIGS. 2B and 2C, tube light 220 comprises a ring of LEDs 225 attached to a light angle compensator 250 disposed within light bulb 235. In the illustrated embodiment, light angle compensator 250 comprises a flexible circular substrate 255 having an adjustment device 265 that passes through the center of the ring of LEDs 225. The adjustment device 265 may be manually or automatically turned in order to adjust the angle of the LEDs 225 with respect to a horizontal surface, such as the floor, as depicted in FIGS. 2B and 2C. The substrate 255 includes a plurality of cutouts 215 dimensioned to hold a diode 225. Referring to FIG. 2A, the light angle 285 can be controlled to achieve an optimal angle in view of light fixture to floor distance. In the illustrated embodiment, the angle 285 is controlled either by turning manual adjustment device 265, or using an automatic adjustment controller that includes a sensor for receiving input from a remote control 145. Such an automatic adjustment controller is depicted in FIG. 4A. In some embodiments, the automatic adjustment controller is used in concert with one or more additional sensors to automatically change the angle of the substrate in response to other conditions such as changes in ambient lighting.

With further reference to FIG. 2, the electromagnetic radiation source 220 is programmable with respect to direction, illumination, retinal area, amplitude, wavelength, and/or spectral property. Alternatively, the electromagnetic radiation source 220 may include a predetermined direction, illumination, retinal area, amplitude, and/or wavelength/spectral character. In the illustrated embodiment, there are three electromagnetic radiation sources 220 separated by a predetermined distance 275, transmitting electromagnetic radiation within a predetermined angle 285, thereby creating an optimal zone 295 of electromagnetic radiation. The distance between the floor and ceiling is indicated as element 290.

Referring to FIGS. 3-4, lighting system 300 includes at least one electromagnetic radiation source 320 comprising a plurality of individual diodes 325 disposed in a number of light tubes 335. In the illustrated embodiment, there are 4 light tubes 335 per radiation source 320 and any number of individual diodes 325 disposed in each tube 335. A first individual diode 325A transmits electromagnetic radiation within a predetermined angle 345A, while a second individual diode 325B transmits electromagnetic radiation within a predetermined angle 345B. In addition, an individual light tube 335A featuring an activated tube light angle compensator 350 transmits electromagnetic radiation within a predetermined angle 360. In this manner, the angle 370 of electromagnetic radiation source 320 increases as the activated tube light angle compensator is activated. Optimal light zone 375 is created by activating the angle compensator, wherein element 380 defines the upper limit and element 385 defines the lower limit.

Referring to FIGS. 4A and 4B, tube light angle compensator 350 comprises a pair of substrates 410A, 410B pivotably attached together at one end, each substrate 410A, 410B including a plurality of cutouts 415 dimensioned to hold a diode 325. The substrates 410A, 410B are disposed within a light tube 335, whereby the angle between the substrates 410A, 410B can be adjusted to control the angle 425 between substrates 410A, 410B, thereby controlling the angle 370 of electromagnetic radiation source 320. The angle 425 between substrates 410A, 410B can be controlled to achieve an optimal angle in view of light fixture to floor distance. In the illustrated embodiment, the angle 425 is controlled either by turning manual adjustment knob 430, or using automatic adjustment controller 450 that includes a sensor 460 for receiving input from a remote control (e.g., remote control 45 of FIG. 1). In some embodiments, the automatic adjustment controller 450 is used in concert with one or more additional sensors to automatically change the angle between substrates 410A, 410B in response to other conditions such as changes in ambient lighting.

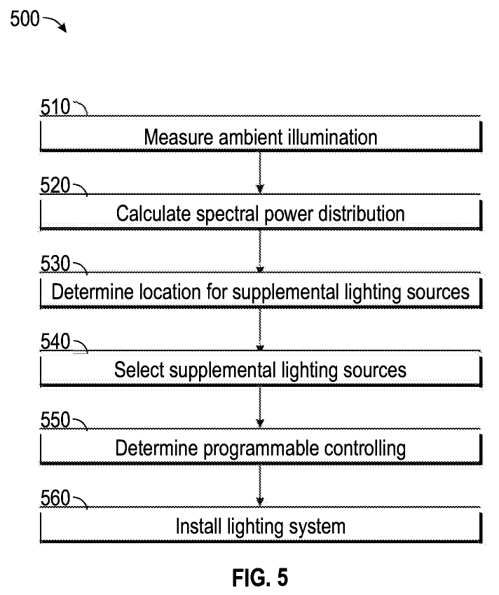

FIG. 5 is a flowchart illustrating a method 500 of designing an optimum lighting system. In particular, operation 510 comprises measuring the ambient illumination to determine the contribution from architectural structures and incident outdoor lighting through windows, skylights, tubes or other means of transmitting outdoor light. In operation 520, the needed spectral power distribution is calculated. In operation 530, the location for placement of the required supplemental lighting sources is determined. In operation 540, the supplemental lighting sources are selected to provide the required SPD, illuminance and MED for the pre-determined eye-planes in the room. Operation 550 entails making a determination for programmable controlling. In operation 560, the system is installed with the respective sensors, sources and programmable controllers.

The electromagnetic radiation systems disclosed herein may be configured to be stable and static. In some embodiments, the electromagnetic radiation system is configured to be programmable and dynamic. An electromagnetic radiation system may be configured as the sole therapeutic element or used in conjunction with spectacle eye-wear or contact lenses. Additionally, the electromagnetic radiation system may be used in conjunction with vision therapy or nutraceutical or pharmaceutical intervention. For example, the spectacle or contact lens may have a refractive correction. The spectacle or contact lens refractive therapy may also include components for off-axis defocus optics and lens filters for regulating the spectral transmission of the lenses.

In some embodiments of the invention, a protective lighting system may be configured for a single eye-plane. One embodiment features a system with programmable electromagnetic radiation sources to provide a desired SPD, MED and Efficacy for the purpose of regulating the growth of the crystalline lens or a region of the retina for a single individual. Alternate embodiments are configured for a plurality of eye-planes. Sensors may be employed to regulate one or more light sources or fixtures to produce the a pre-determined SPD and MED at each eye plane.

In further embodiments, a protective lighting system may incorporate light sources disposed on or within a display such as a computer display or a hand held display. In some configurations, the light source can be added to the display. In other configurations, the light source is used to modulate the output of the display.

In one embodiment, the electromagnetic radiation sources are individually programmed to provide a different SPD and MED to different eye-planes for the purpose of modulating the growth factors of individual eyes. The electromagnetic radiation sources are selected for their spectral properties and are configured to provide a pre-determined direction and area of radiation.

In another embodiment, an eye-plane space is configured with a sensor to measure the SPD and MED These data may be incorporated into a computer program product which in turn regulates the amplitude, direction, area or spectral output of the electromagnetic radiation sources in the system.

In yet another embodiment, the electromagnetic radiation refractive therapy system may be configured with a sensor to measure blood serum level of Vitamin D. This sensor may be implanted or designed as a non-invasive sensor. These data may be incorporated into a computer program product which in turn regulates the amplitude, direction, area or spectral output of the electromagnetic radiation sources in the system to regulate the minimal erythema doses of the system. By way of example, in one embodiment the SPD for daylight includes a correlated color temperature (CCT) of 5500K, an Efficacy of 50 lumens per watt, and 1.0 minimal erythema doses with an 8 hour exposure.

One embodiment of the invention comprises a lighting system for the control of the progression of myopia of an eye, comprising at least one light source which produces: (i) an illuminance greater than 3500 lux; (ii) a correlated color temperature greater than 3500 K; (iii) a spectral band width from 320 nm to 680 nm; and (iv) a minimal erythema dose of 0.5 with 8 hours of exposure at the plane of the eye.

Another embodiment of the invention comprises a lighting system for the reduction of hyperopia of an eye, comprising at least one light source which produces: (i) an illuminance less than 1500 lux; (ii) a color temperature less than 3500 K; and (iii) a spectral band width from 450 nm to 820 nm.

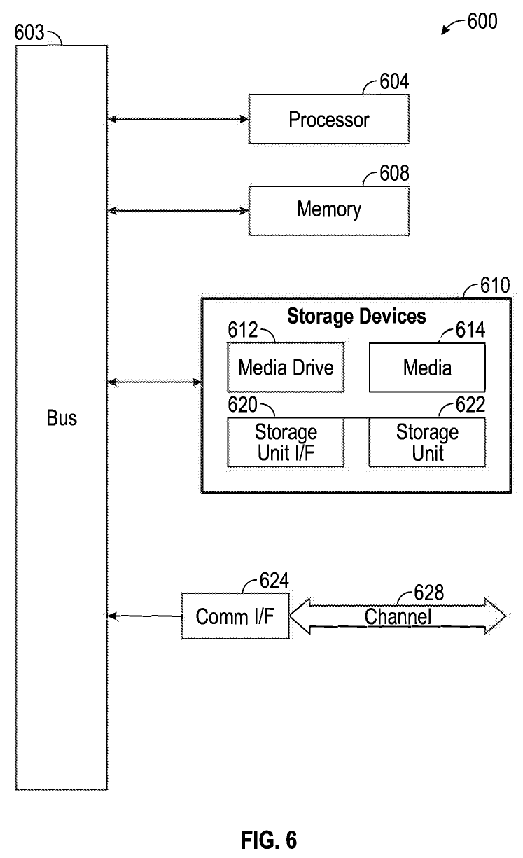

As used herein, the term “module” might describe a given unit of functionality that can be performed in accordance with one or more embodiments of the present application. As used herein, a module might be implemented utilizing any form of hardware, software, or a combination thereof. For example, one or more processors, controllers, ASICs, PLAs, PALs, CPLDs, FPGAs, logical components, software routines or other mechanisms might be implemented to make up a module. In implementation, the various modules described herein might be implemented as discrete modules or the functions and features described can be shared in part or in total among one or more modules. In other words, as would be apparent to one of ordinary skill in the art after reading this description, the various features and functionality described herein may be implemented in any given application and can be implemented in one or more separate or shared modules in various combinations and permutations. Even though various features or elements of functionality may be individually described or claimed as separate modules, one of ordinary skill in the art will understand that these features and functionality can be shared among one or more common software and hardware elements, and such description shall not require or imply that separate hardware or software components are used to implement such features or functionality.

Where components or modules of the application are implemented in whole or in part using software, in one embodiment, these software elements can be implemented to operate with a computing or processing module capable of carrying out the functionality described with respect thereto. One such example of a computing module is shown in FIG. 6. Various embodiments are described in terms of this example-computing module 600. After reading this description, it will become apparent to a person skilled in the relevant art how to implement embodiments of the application using other computing modules or architectures.

Referring now to FIG. 6, computing module 600 may represent, for example, computing or processing capabilities found within desktop, laptop and notebook computers; hand-held computing devices (PDA's, smart phones, cell phones, palmtops, etc.); mainframes, supercomputers, workstations or servers; or any other type of special-purpose or general-purpose computing devices as may be desirable or appropriate for a given application or environment. Computing module 600 might also represent computing capabilities embedded within or otherwise available to a given device. For example, a computing module might be found in other electronic devices such as, for example, digital cameras, navigation systems, cellular telephones, portable computing devices, modems, routers, WAPs, terminals and other electronic devices that might include some form of processing capability.

Computing module 600 might include, for example, one or more processors, controllers, control modules, or other processing devices, such as a processor 604. Processor 604 might be implemented using a general-purpose or special-purpose processing engine such as, for example, a microprocessor, controller, or other control logic. In the illustrated example, processor 604 is connected to a bus 603, although any communication medium can be used to facilitate interaction with other components of computing module 600 or to communicate externally.

Computing module 600 might also include one or more memory modules, simply referred to herein as main memory 608. For example, preferably random access memory (RAM) or other dynamic memory, might be used for storing information and instructions to be executed by processor 604. Main memory 608 might also be used for storing temporary variables or other intermediate information during execution of instructions to be executed by processor 604. Computing module 600 might likewise include a read only memory (“ROM”) or other static storage device coupled to bus 603 for storing static information and instructions for processor 604.

The computing module 600 might also include one or more various forms of information storage mechanism 610, which might include, for example, a media drive 612 and a storage unit interface 620. The media drive 612 might include a drive or other mechanism to support fixed or removable storage media 614. For example, a hard disk drive, a floppy disk drive, a magnetic tape drive, an optical disk drive, a CD, DVD or Blu-ray drive (R or RW), or other removable or fixed media drive might be provided. Accordingly, storage media 614 might include, for example, a hard disk, a floppy disk, magnetic tape, cartridge, optical disk, a CD, DVD or Blu-ray, or other fixed or removable medium that is read by, written to or accessed by media drive 612. As these examples illustrate, the storage media 614 can include a non-transitory computer readable medium having computer executable program code embodied thereon.

In alternative embodiments, information storage mechanism 610 might include other similar instrumentalities for allowing computer programs or other instructions or data to be loaded into computing module 600. Such instrumentalities might include, for example, a fixed or removable storage unit 622 and an interface 620. Examples of such storage units 622 and interfaces 620 can include a program cartridge and cartridge interface, a removable memory (for example, a flash memory or other removable memory module) and memory slot, a PCMCIA slot and card, and other fixed or removable storage units 622 and interfaces 620 that allow software and data to be transferred from the storage unit 622 to computing module 600.

Computing module 600 might also include a communications interface 624. Communications interface 624 might be used to allow software and data to be transferred between computing module 600 and external devices. Examples of communications interface 624 might include a modem or softmodem, a network interface (such as an Ethernet, network interface card, WiMedia, IEEE 802.XX or other interface), a communications port (such as for example, a USB port, IR port, RS232 port Bluetooth® interface, or other port), or other communications interface. Software and data transferred via communications interface 624 might typically be carried on signals, which can be electronic, electromagnetic (which includes optical) or other signals capable of being exchanged by a given communications interface 624. These signals might be provided to communications interface 624 via a channel 628. This channel 628 might carry signals and might be implemented using a wired or wireless communication medium. Some examples of a channel might include a phone line, a cellular link, an RF link, an optical link, a network interface, a local or wide area network, and other wired or wireless communications channels.

In this document, the terms “computer program medium” and “computer usable medium” are used to generally refer to media such as, for example, memory 608, storage unit 620, media 614, and channel 628. These and other various forms of computer program media or computer usable media may be involved in carrying one or more sequences of one or more instructions to a processing device for execution. Such instructions embodied on the medium, are generally referred to as “computer program code” or a “computer program product” (which may be grouped in the form of computer programs or other groupings). When executed, such instructions might enable the computing module 600 to perform features or functions of the present application as discussed herein.

While various embodiments of the present application have been described above, it should be understood that they have been presented by way of example only, and not of limitation. Likewise, the various diagrams may depict an example architectural or other configuration for the disclosure, which is done to aid in understanding the features and functionality that can be included in the disclosure. The application is not restricted to the illustrated example architectures or configurations, but the desired features can be implemented using a variety of alternative architectures and configurations. Indeed, it will be apparent to one of skill in the art how alternative functional, logical or physical partitioning and configurations can be implemented to implement the desired features of the present application. Also, a multitude of different constituent module names other than those depicted herein can be applied to the various partitions. Additionally, with regard to flow diagrams, operational descriptions and method claims, the order in which the steps are presented herein shall not mandate that various embodiments be implemented to perform the recited functionality in the same order unless the context dictates otherwise.

Although the application is described above in terms of various exemplary embodiments and implementations, it should be understood that the various features, aspects and functionality described in one or more of the individual embodiments are not limited in their applicability to the particular embodiment with which they are described, but instead can be applied, alone or in various combinations, to one or more of the other embodiments of the disclosure, whether or not such embodiments are described and whether or not such features are presented as being a part of a described embodiment. Thus, the breadth and scope of the present application should not be limited by any of the above-described exemplary embodiments.

Terms and phrases used in this document, and variations thereof, unless otherwise expressly stated, should be construed as open ended as opposed to limiting. As examples of the foregoing: the term “including” should be read as meaning “including, without limitation” or the like; the term “example” is used to provide exemplary instances of the item in discussion, not an exhaustive or limiting list thereof; the terms “a” or “an” should be read as meaning “at least one,” “one or more” or the like; and adjectives such as “conventional,” “traditional,” “normal,” “standard,” “known” and terms of similar meaning should not be construed as limiting the item described to a given time period or to an item available as of a given time, but instead should be read to encompass conventional, traditional, normal, or standard technologies that may be available or known now or at any time in the future. Likewise, where this document refers to technologies that would be apparent or known to one of ordinary skill in the art, such technologies encompass those apparent or known to the skilled artisan now or at any time in the future.

The presence of broadening words and phrases such as “one or more,” “at least,” “but not limited to” or other like phrases in some instances shall not be read to mean that the narrower case is intended or required in instances where such broadening phrases may be absent. The use of the term “module” does not imply that the components or functionality described or claimed as part of the module are all configured in a common package. Indeed, any or all of the various components of a module, whether control logic or other components, can be combined in a single package or separately maintained and can further be distributed in multiple groupings or packages or across multiple locations.

Additionally, the various embodiments set forth herein are described in terms of exemplary block diagrams, flow charts and other illustrations. As will become apparent to one of ordinary skill in the art after reading this document, the illustrated embodiments and their various alternatives can be implemented without confinement to the illustrated examples. For example, block diagrams and their accompanying description should not be construed as mandating a particular architecture or configuration.

1. A method for the regulation of the development of ocular refractive errors, comprising:
controlling at least one light source using a processor, the at least one light source emitting an electromagnetic radiation variable with respect to one or more of a direction, an illuminance, a retinal area, an amplitude, a wavelength, and a spectral output; and
regulating the at least one light source and producing a spectral power distribution at a plane of an eye;
wherein the one or more of the direction, the illuminance, the retinal area, the amplitude, the wavelength, and the spectral output of the electromagnetic radiation emitted by the at least one light source is determined by the processor in response to an input from at least one sensor such that the at least one light source achieves an illuminating range from the at least one light source to a surface to provide the electromagnetic radiation to the plane of the eye for regulating a development of refractive errors of the eye, and wherein the spectral power distribution is based on the spectral output determined by the processor.

2. The method of claim 1, wherein the illuminance is greater than 3500 lux.

3. The method of claim 1, wherein the illuminance is greater than 1500 lux.

4. The method of claim 1, wherein the spectral power distribution comprises a spectral band width from 320 nm to 680 nm.

5. The method of claim 1, wherein the light source is selected from the group consisting of: LEDs, incandescent lighting, fluorescent lighting, compact fluorescent lighting, metal halide lighting, ceramic metal halide lighting, mercury vapor lighting, and xenon lighting.

6. The method of claim 1, wherein the light source is configured to produce radiation having a predetermined amplitude, spectral power distribution, and minimal erythema doses from the ultraviolet spectral contribution, at the plane of the eye.

7. A method for the control of the progression of myopia of an eye, comprising:
controlling at least one light source controlled using a processor;
regulating the light source and producing a pre-determined spectral power distribution at a plane of the eye;
wherein the light source produces an illuminance such that the light source achieves an illuminating range from a light fixture to a surface to provide an electromagnetic radiation to a plane of the eye for regulating a development of refractive errors of the eye,
wherein the light source is variable with respect to the illuminance.

8. The method of claim 7, wherein a minimal erythema dose of the light source is 0.5 with 8 hours of exposure at the plane of the eye.

9. A method for the reduction of hyperopia of an eye, comprising:
controlling at least one light source using a processor;
regulating the light source and producing a pre-determined spectral power distribution at a plane of the eye;
wherein the at least one light source produces:
a correlated color temperature less than 3500 K;
an illuminance less than 1500 lux; and
a spectral band width from 450 nm to 820 nm.

10. The method of claim 9, wherein the light source is selected from the group consisting of: one or more LEDs, incandescent lighting, fluorescent lighting, compact fluorescent lighting, metal halide lighting, ceramic metal halide lighting, mercury vapor lighting, xenon lighting.
